(19) United States
(12) Patent Application Publication
(10) Pub. No.: US 2021/0353794 A1
(43) Pub. Date: Nov. 18, 2021

(54) Method and device to allow antiseptic proximity seating

(71) Applicants: Victor Popa-Simil, Los Alamos, NM (US); Ioana Livia Popa-Simil, Ann Arbor, MI (US); Andrei Popa-Simil, Albuquerque, NM (US); Liviu Popa-Simil, Los Alamos, NM (US); Gabriel Vasilescu, Ottawa (CA); Adrian Boboc, Tlmisoara (RO); Arpad Harangozo, Bucharest (RO)

(72) Inventors: Victor Popa-Simil, Los Alamos, NM (US); Ioana Livia Popa-Simil, Ann Arbor, MI (US); Andrei Popa-Simil, Albuquerque, NM (US); Liviu Popa-Simil, Los Alamos, NM (US); Gabriel Vasilescu, Ottawa (CA); Adrian Boboc, Tlmisoara (RO); Arpad Harangozo, Bucharest (RO)

(73) Assignee: LAVM LLC, Los Alamos, NM (US)

(21) Appl. No.: 16/876,337

(22) Filed: May 18, 2020

(51) Int. Cl. A61L 2/10; B25J 9/16; F24H 3/00; G06K 9/00; G05D 1/00

(52) U.S. Cl. CPC A61L 2/10; B25J 9/1615; F24H 3/002; G06K 9/00335; G05D 2201/0206; G05D 1/0027; A61L 2202/121; A61L 2202/15; A61L 2202/25; G06K 9/00362

(57) ABSTRACT

With respect to pandemics, restaurants, stadiums, cafeterias, and bars are among the most dangerous places to socialize due to the form of interaction. Exchange of bacteria and viruses are facilitated intensively from the mouth and nose via air transport in droplets of saliva and bodily fluids being deposited on surfaces, foods, and in the air. The present development creates a comfortable environment where the air-space of each customer is separated by invisible cold-hot air and vis-UV barriers, which take away bacteria and viruses, destroying them and preventing recirculation while offering pleasant, clean, and sanitized ambient conditions for a face to face meeting. An immersive experience can be provided with surrounding screens and gas therapy that may be customized according to the customer's preferences. Food distribution is performed with a robotic shuttle system and when the customer departs, an extensive cleanup is performed using chemicals and UV irradiation through robotic systems.

With respect to pandemics, restaurants, stadiums, cafeterias, and bars are among the most dangerous places to socialize due to the form of interaction. Exchange of bacteria and viruses are facilitated intensively from the mouth and nose via air transport in droplets of saliva and bodily fluids being deposited on surfaces, foods, and in the air. The present development creates a comfortable environment where the air-space of each customer is separated by invisible cold-hot air and vis-UV barriers, which take away bacteria and viruses, destroying them inside catalytic organic fractions burner, heat and vaporization, condensation or desiccation and preventing them from recirculation or transfer while offering pleasant, clean, and sanitized ambient conditions for a face to face meeting. The process combines a pseudo-virga effect with atmospheric boxing in order to assure a redundant protection layers and comfort. An immersive experience can be provided with surrounding screens and gas therapy that may be customized according to the customer's preferences. Food distribution is performed with a robotic shuttle system and when the customer departs, an extensive cleanup is performed using chemicals and UV irradiation through robotic systems. In addition, the table surfaces are layered with photo-active nano-coating that presents enhanced antiseptic properties. All furniture is designed with round accessible surfaces, easily visible, and washable. The entire structure is conceived as booth-modules, in order to be easily assembled in a large variety of shapes and structures, containing autonomous thermo-mechanical systems. The design is intended to make maintenance more affordable and efficient along with being reconfigurable depending on necessity. The structure is also produced as seats with reduced table or no table to equip conference rooms, stadiums, theatres, bars, etc. insuring about the same. Unexpected incidents such as coughing and sneezing are prevented by an electronic monitoring system that detects the incident and, in real time, shoots several high pressure gas puffs to interfere and combine into a short opposing local wind able to stop larger airborne droplets, preventing them from penetrating the nearby air space.

### STATEMENT REGARDING FEDERALLY SPONSORED R&D

This invention was made with NO Government support.

### NAMES OF PARTIES TOA JOINT RESEARCH AGREEMENT

This work was part of research of the mentioned inventors.

### CROSS REFERENCE TO RELATED APPLICATIONS

This application claims no priority.

### BACKGROUND OF THE INVENTION

### 1. Field of the Invention

The present invention relates to a method and device to allow safe face to face meetings for eating, drinking, and talking in comfortable conditions even in the presence of a pandemic with airborne transmission capabilities and high communicability. The invention utilizes boxed airspaces and onsite air decontamination procedures.

A well-documented example is given by Jianyun Lu et al. in the paper “COVID-19 Outbreak Associated with Air Conditioning in Restaurant, Guangzhou, China, 2020”, where he documents that during Jan. 26-Feb. 10, 2020, an outbreak of the 2019 novel coronavirus disease in an air-conditioned restaurant in Guangzhou, China, infected 3 family clusters. The airflow direction was consistent with the droplet transmission. To prevent the spread of the virus in restaurants, he recommends increasing the distance between tables and improving ventilation. Based on our knowledge however, that is not enough for virus propagation denial, and too much of a reduction of capacity of such places causes them to lose profit. To overcome these obstacles, we came with a technological solution to mitigate these divergent aspects.

In fact “From January 26 through Feb. 10, 2020, an outbreak of 2019 novel coronavirus disease (COVD-19) affected 10 persons from 3 families (families A-C) who had eaten at the same air-conditioned restaurant in Guangzhou, China. One of the families had just traveled from Wuhan, Hubei Province, China. We performed a detailed investigation that linked these 10 cases together . . . . On Jan. 23, 2020, family A traveled from Wuhan and arrived in Guangzhou. On January 24, the index case-patient (patient A1) ate lunch with 3 other family members (A2-A4) at restaurant X. Two other families, B and C, sat at neighboring tables at the same restaurant. Later that day, patient A1 experienced onset of fever and cough and went to the hospital. By February 5, a total of 9 others (4 members of family A, 3 members of family B, and 2 members of family C) had become ill with COVID-19.

The only known source of exposure for the affected persons in families B and C was patient A1 at the restaurant. We determined that virus had been transmitted to >1 member of family B and >1 member of family C at the restaurant and that further infections in families B and C resulted from within-family transmission. Thumbnail of Sketch showing arrangement of restaurant tables and air conditioning airflow at site of outbreak of 2019 novel coronavirus disease, Guangzhou, China, 2020. Red circles indicate seating of future case-patients; yellow-filled red circle indicates index case-patient. Sketch showing arrangement of restaurant tables and air conditioning airflow at site of outbreak of 2019 novel coronavirus disease, Guangzhou, China, 2020. Red circles indicate seating of future case-patients; yellow-filled red.”, attached as FIG. 1.

We understood this issue, and consider the Chinese specialists not good enough, because we also saw cases of longer distance propagations via AC recycling systems, deriving to large number of infestations on airplane, cruise ships, homes, and hospitals. Our approach to make meetings among many people safe is to use advanced technologies in order deal with each aspect of virus propagation, survival, and transmission.

### 2. Description of the Prior Art

There seems to be no specific prior art we may consider as direct reference, but we may consider prior art related to anti-bacteriological protection in high risk research laboratories, with bio-hazard level 4 where autonomous respiratory gas and full body PPE (personal protective equipment) are used to assure an individual protection factor of 99.99%

All the actual solutions that aim to prevent the virus transmission are based on covering the mouth and nose, which makes eating, drinking nearly impossible with conversing being extremely difficult.

The presently available suits are difficult to dress on and off, and during this procedure the highest contamination risk is high. Another way to stop contamination from spreading is to use a similar design to anti-bioterrorism units with separate ventilation circuits. In these units, the bio-hazardous air and fluids are eliminated via an incinerator, plasma burner, or chemical scrubber. The system we propose is a modular open system, and any supplementary required function may be added, or its complexity may be reduced as necessary.

### BRIEF DESCRIPTION OF THE DRAWINGS

FIG. 1—is a schematic view of the restaurant X, incident showing the arrangement of restaurant tables and air conditioning airflow at site of outbreak of 2019 novel coronavirus disease, Guangzhou, China, 2020, January 23;

FIG. 2—A chart showing the exhaled water droplets distribution, vs. filter retention capabilities.

FIG. 3 shows the actual state of the art with respect to parties and socializing eating sessions

FIG. 4 details on table module

FIG. 5 describes the a cubic table for 4 person system

FIG. 6 gives details on a hexagonal table equivalent,

FIG. 7 describes a restaurant floor made of 8 hexagonal structures connected inside a hall;

FIG. 8 describes an octagonal table arrangement

FIG. 9 shows a section through a customer sitting at a cafeteria table

FIG. 10—an application of the airborne droplets shielding in the actual restaurant environment

FIG. 11—Schematic diagram of a stadium seat pair:

FIG. 12—bar seats improvement

### FIGURES DETAILS

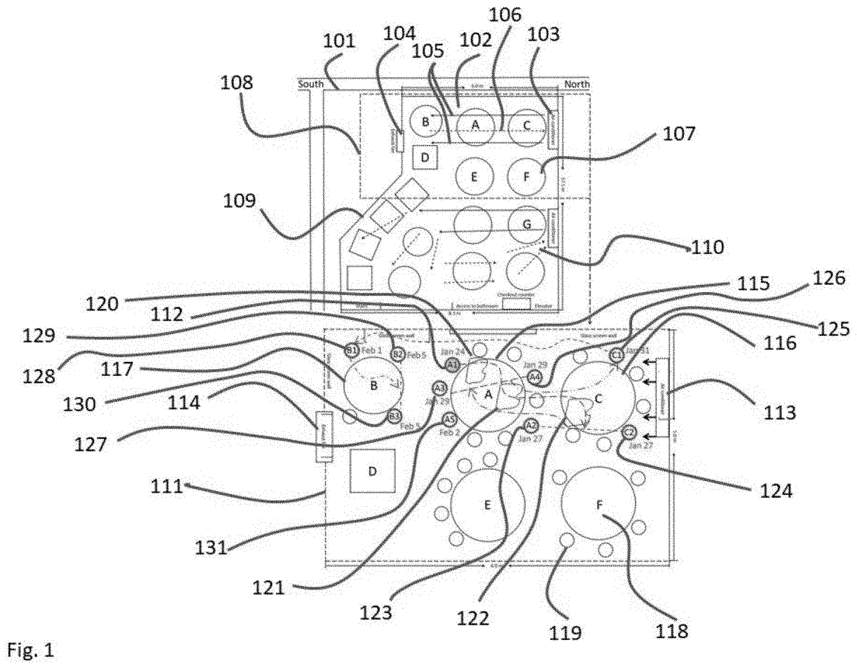

FIG. 1—is a schematic view of the restaurant X, incident showing the arrangement of restaurant tables and air conditioning airflow at site of outbreak of 2019 novel coronavirus disease, Guangzhou, China, 2020, January 23;
101—Restaurant hall walls;
102—Restaurant interior 3 table row on west side;
103—North wall air conditioning unit;
104—South wall air conditioning units;
105—Air flow lines from N to S;
106—Reverse air flow from S to N inside the room;
107—Second row of tables unaffected;
108—The area of interest where COV transmission occurred;
109—The other half of restaurant hall where was no transmission;
110—Air flow currents inside the second half of restaurant hall;
111—Area of interest in a detailed diagram;
112—The infected, contagious person A1, who was declared sick, next day on January 24;
113—N wall AC unit;
114—S wall AC unit;
115—Central table “A” on W side of the hall;
116—SW table “B”;
117—NW table “C”;
118—Central row, nearby, “E” and “F”;
119—Un-contaminated people on seats near-by;
120—Client “A1” exhaust plume just exhaled;
121—Previously exhaled aerosol droplets contaminated plume.
122—Previously exhaled plume, driven by air currents at table “C”;
123—Family's “A” member “A2”, infected and found positive in 4 days;
124—Family's “C” member “C2”, infected and found positive in 4 days, on January 27;
125—Family's “C” member “C1”, infected and found positive in 8 days, on January 31;
126—Family's “A” member, “A4” Infected and found positive in 6 days, on January 29;
127—Family's “A” member “A3” infected and found positive in 6 days, on January 29;
128—Family's “B” member “B1” infected and found positive after 9 days, on February 1;
129—Family's “B” member “B2” infected and positive after 13 days, on February 5^{th};
130—Family's “B” member “B3” infected and positive after 13 days on February 5^{th};

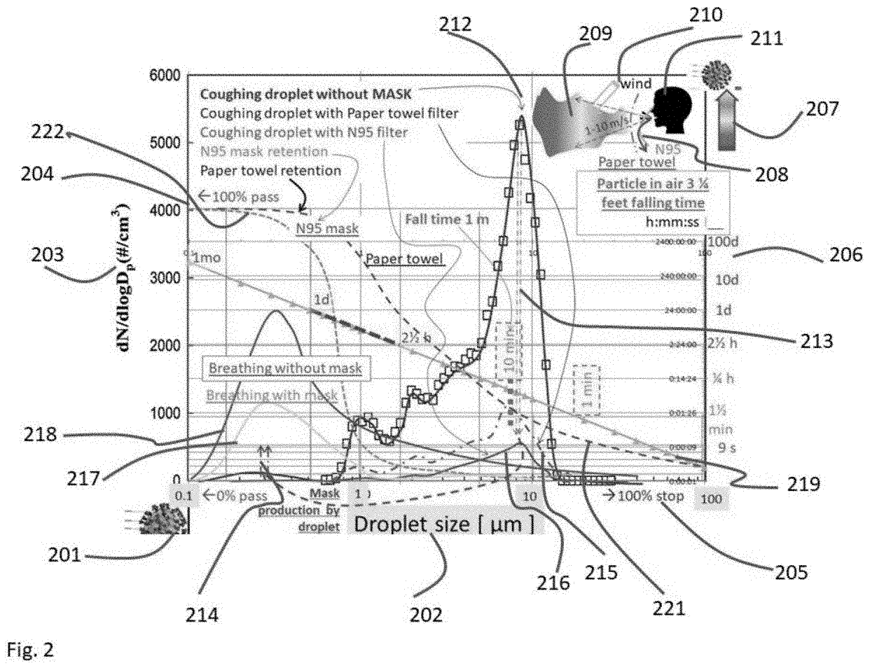

FIG. 2—A chart showing the exhaled water droplets distribution, vs. filter retention capabilities.
201—COVID-19 average dimension of 100 nm;
202—Droplet size scale from 0.1-100 microns;
203—Number of particles per cc as function of particle magnitude;
204—Particle filter 100% pass upper limit;
205—Particle filter 0% pass=100% stopping power, lower limit;
206—Time airborne droplets with 1.5 g/cc density float in air;
207—Concentration color code for virus inside an air exhalation;
208—Exhaled air-jet change of direction dur to mouth cover;
209—Exhalation plume without any face cover;
210—Wind direction;
211—Head of a contagious person;
212—Dimensional distribution of airborne droplets while coughing openly;
213—Retention in a new N95 mask, first shout;
214—Conversion of large droplets into small droplets released by mask by atomization;
215—Dimensional distribution of droplets released through a paper-towel filter;
216—Release of droplets spectrum through an N95 mask (without exhaust valve);
217—Normal breathing and air exhalation through a mask;
218—Dimensional distribution of droplets during an open exhalation;
219—The time it takes a droplet to free-fall 1 m (3.3 ft) based on Stokes force;
221—Paper towel filter retention curve;
222—N95 filter retention curve

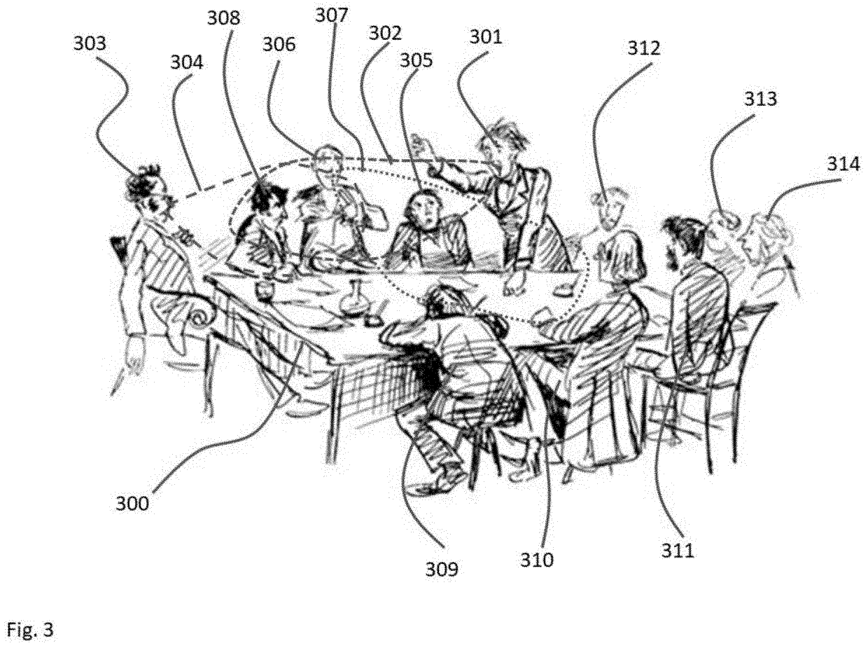

FIG. 3 shows the actual state of the art with respect to parties and socializing eating sessions
300—Table
301—Effervescent speaker;
302—Droplets plume exhaled during effervescent speech;
303—Arguing speaker;
304—Arguing speaker plume;
305—Person listening;
306—Person arguing over the table;
307—Plume over the table;
308—Person listening;
309—Person eating;
310—person arguing back over the table;
311—Person amazed by parallel talks;
312—Bored person;
313—Ecstatic person;
314—Old lady listening;

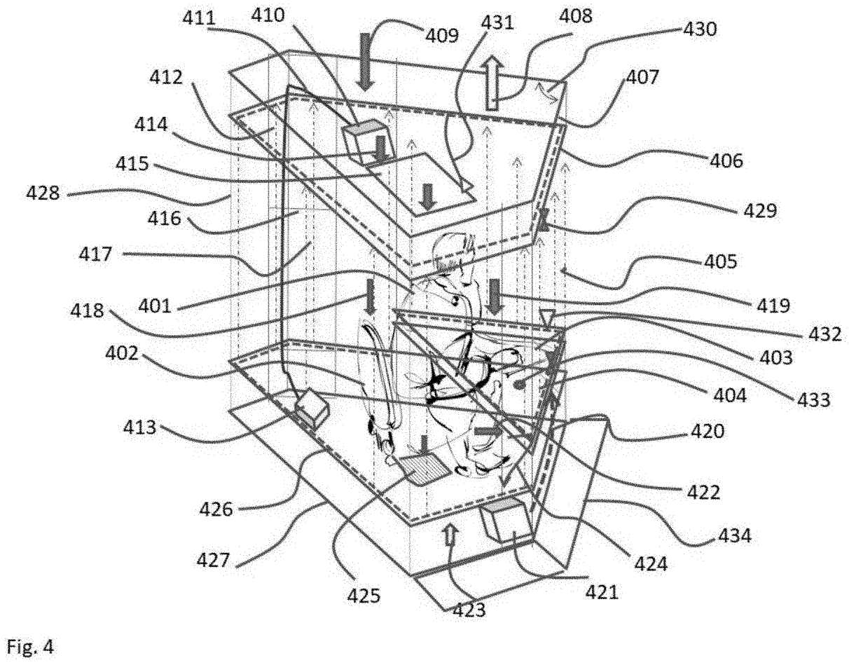

FIG. 4 details on table module:
401—Customer sitting on a table section
402—Ergonomic chair;
403—Table top;
404—Table lateral protective structure, with hot air nozzle and UV retro-reflector;
405—Hot air ascendant lamellar jet;
406—Hot air absorption holes
407—Double layer containing collection tubes;
408—Hot air exhaust;
409—Fresh intake airflow;
410—Input air processing unit (filter, AC, humidity, composition)
411—Heat-pipe assembly;
412—Border region hot air laminar flow;
414—Air cooler unit;
415—Descendent cold air vortex celling hole and vent;
416—Internal wall for dress, coat hanger;
417—Warm air jet, for coat decontamination;
418—Descendent air-flow over customer;
419—Descendent airflow in front of customer;
420—Hot air flow internal tubing;
421—Heat exchanger and catalytic burner hot air cleaner;
422—Used cold air intake;
423—Hot air exit ducts and nozzles;
424—Used cold air ducts inside walls and fake floor;
425—Central, under-chair used cold-air floor intake;
426—Hot air perimeter nozzles and UV corner—back-reflector;
427—Lower floor plate;
428—Transparent, opaque or video screen separator;
429—UV chain LEDs;
430—Tip angle or profile;
431—Compressed air puff generator;
432—Hot compressed air puff generator;
433—Microphone/loudspeaker pulsed phased array;
434—Lateral board.

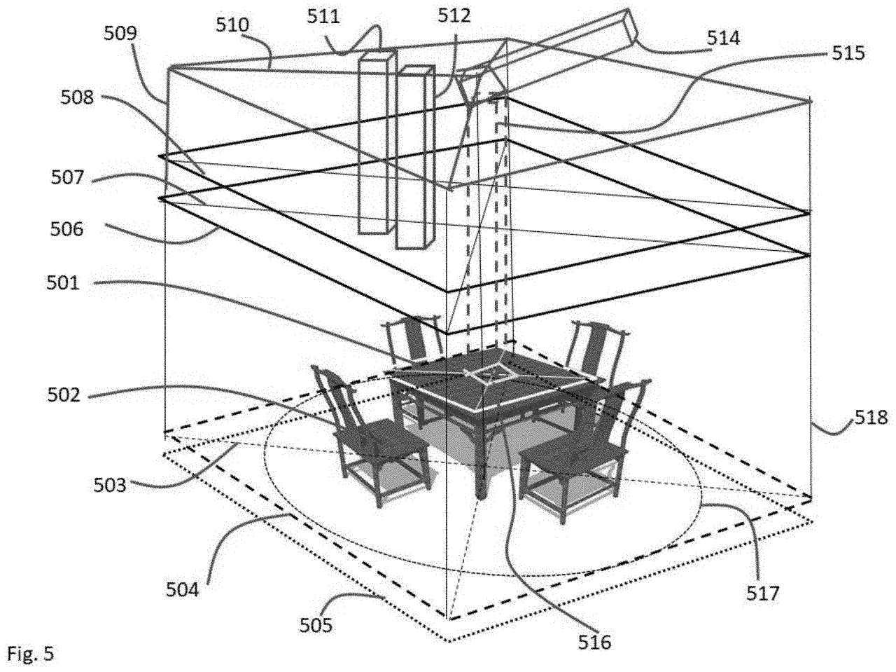

FIG. 5 describes the cubic table for a 4 person system
501—Four seats equivalent table;
502—Chair;
503—Diagonal separation zone;
504—Lateral border zone;
505—Bottom floor layer;
506—Celling layer;
507—Diagonal separation and protection zone;
508—Lateral upper celling zone;
509—Sliding lateral separation screen with multiple function;
510—Diagonal, sliding separation screen;
511—Fresh air input duct;
512—Hot air output duct;
514—Food delivery robotic access duct;
515—Vertical access sliding duct;
516—Table protection limit and central access assembly;
517—First ring of protection;
518—Vertical pillars structure support.

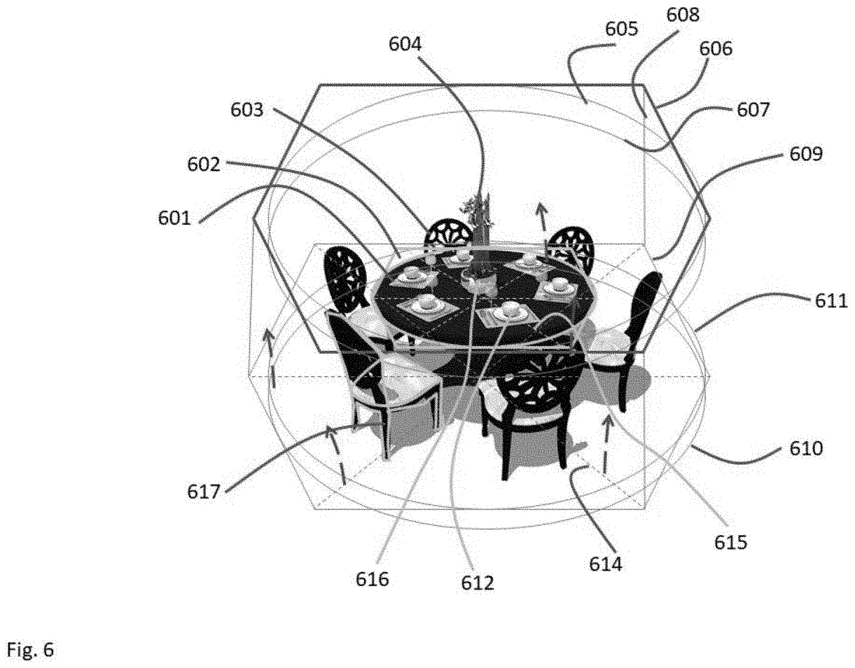

FIG. 6 gives details on a hexagonal table equivalent,
601—60 deg. module booth with round border;
602—Module booth with table having a linear edge
603—Chair;
605—Ornamental plant;
607—Upper ceiling round cover;
608—Lateral pillar;
609—Hexagonal boundary, upper floor;
610—Round lower floor;
611—Round upper floor with hot air and UV shield;
612—Dish placed in wrong position;
614—Lateral separator and hot air, UV screen;
615—Table top separator and hot air, UV screen;
616—Central table dish or distribution robot;
617—Chair placed in wrong position.

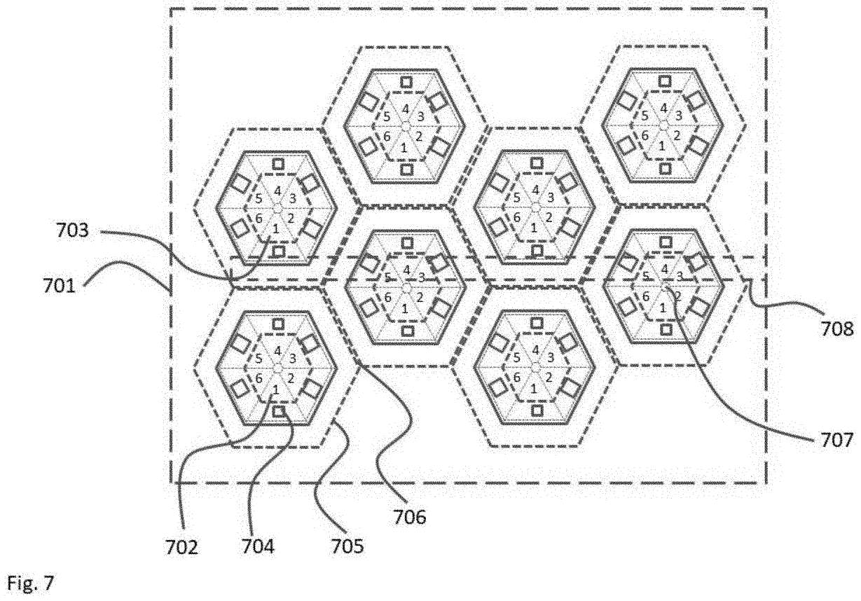

FIG. 7 describes a restaurant floor made of 8 hexagonal structures connected inside a hall;
701—Restaurant hall;
702—Table module as a 60 deg. triangular prism booth on first row;
703—Table module as a 60 deg. triangular prism booth on second row;
704—Customer 2; 1 seat;
705—Hexagonal boundary with protective shields:
706—Joint between two hexagonal tables;
707—Central table spot that may be used for robotic food and drinks delivery;
708—Robot access ducts.

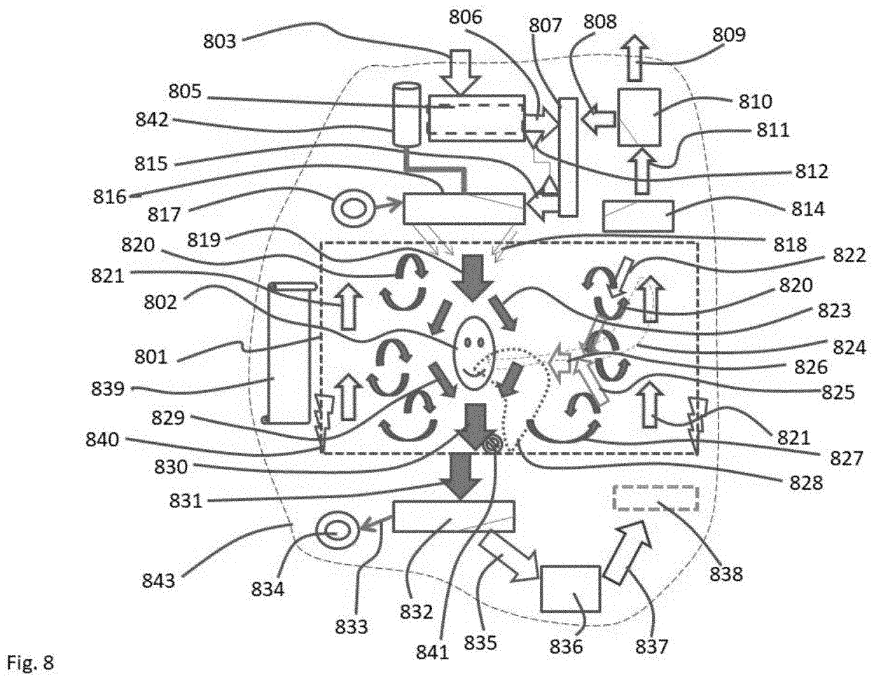

FIG. 8 describes the air flow and protective assemblies inside the seat-booth
801—atmospheric box where the customer is seated;
802—Customer's head as main source of hazardous airborne effluents;
803—Air intake from atmosphere;
804—Symmetry separation line;
805—Air intake filtering system and HEPA filter;
806—Air output in gas/gas heat exchanger;
807—Catalytic organic matter burner and CO₂ sequester;
808—Recirculating air optional input;
809—Hot air exhaust;
810—Hot air filter and catalytic burner;
811—Hot air intake;
812—Heat pipe;
814—AC unit, cold side;
815—Cold air exit from carbon dioxide arrester and input into AC unit cold side;
816—AC unit cooler;
817—Recycled water condenser;
818—Ultrasound atomizer, water droplets insertion in AV vent;
819—Celling cold air vent, down coming airflow;
820—Air vortex;
821—Hot air up-flow coming from hot air boundary nozzle;
822—Cold air puff to stop sneezing exhaled jet;
823—Down coming cold airflow;
824—Sneezing water droplets penetration through down going cold airflow;
825—Cold air puff to stop sneezing exhaled jet;
826—Constructive interference anti-sneeze puff;
827—Down on floor or table top vortex;
828—Sneeze or exhalation airborne droplets plume;
829—Down going cold airflow;
830—Outgoing cold airflow through floor vent;
831—Airflow entering the air cooler-desiccator unit;
832—Cooler-desiccator unit followed by air heater;
833—Water condensate drain to vaporizer;
834—Vaporizer eater transfer pipe to recycled water condenser 817;
835—Heated air enters the catalytic burner;
836—Organic compounds catalytic burner;
837—Hot air exit from catalytic burner through boundary nozzles;
838—Floor boundary nozzles for forming a hot airflow curtain;
839—Boundary retractable multi-purpose screens;
840—UV-C sterilizing curtain reflected on corner reflectors surface;
841 —Microphone loud speaker system for multipurpose action;
842—Multi gas tubes for multipurpose action;
843—Seat-table modular booth assembly.

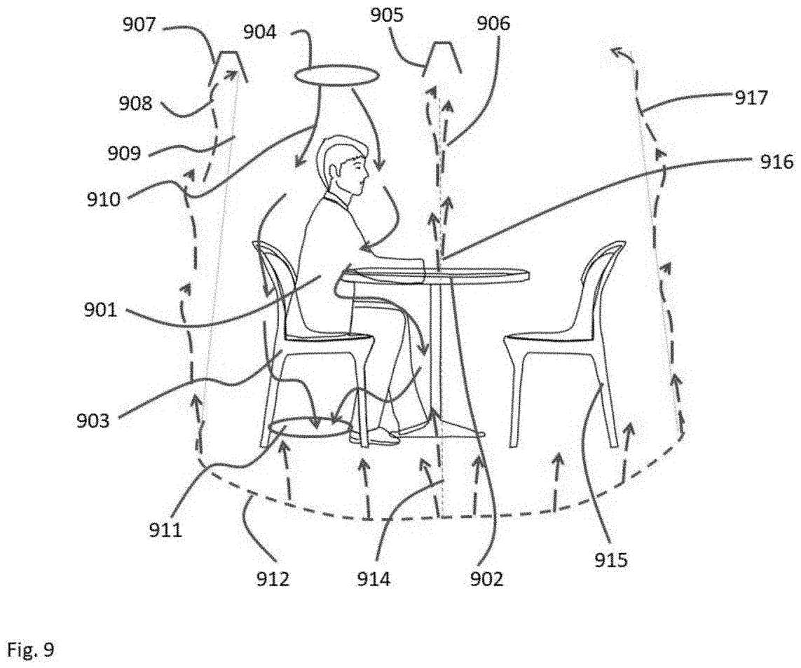

FIG. 9 shows a section through a customer sitting at a cafeteria table
901—Customer sitting;
902—Table;
903—Chair;
904—Upper cold air vent;
905—Upper-central hot air absorption nozzle;
906—Ascending hot air;
907—Upper-lateral ascending hot air absorption nozzle;
908—Lateral hot air shield ascending current;
909—UV shield;
910—Descending cold air with aerosol ultra-sound humidifier;
911—Cold air bottom intake vent;
912—Perimeter hot-air and UV shield;
914—Central hot-air and UV shield;
915—Opposite chair;
916—Central table ascending hot-air and UV shield;
917—Lateral, back of opposite chair hot-air and UV shield;

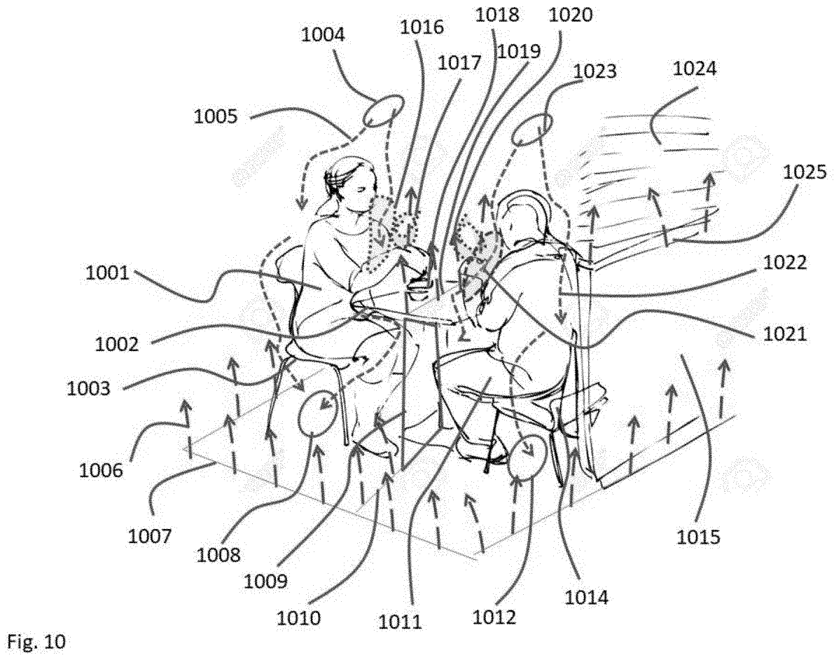

FIG. 10—an application of the airborne droplets shielding in the actual restaurant environment
1001—Woman sitting at a table;
1002—Table top;
1003—Chair on left;
1004—Cold air upper vent;
1005—Cold air down-current;
1006—Hot air ascendant current;
1007—Perimeter hot air nozzle;
1008—Cold air intake vent;
1009—Table additional vertical separator;
1010—Separator floor hot-air nozzle;
1011—Man sitting at table;
1012—Cold-air intake vent;
1014—Chair on the right;
1015—Booth for two separation screen;
1016—Woman's exhaled gas plume trained downwards by cold air flow;
1017—Woman's exhale plume trained upwards by hot air flow;
1018—Coffee's vapors trained upwards and vortexing;
1019—Table-top hot-air nozzle and UV screen;
1020—Man's exhale plume trained upwards by hot air flow;
1021—Man's exhaled plume pushed downwards by cold air flow;
1022—Descending cold air flow;
1023—Cold air upper vent;
1024—Supplementary privacy screen;
1025—Hot air nozzle and UV shield at back screen;

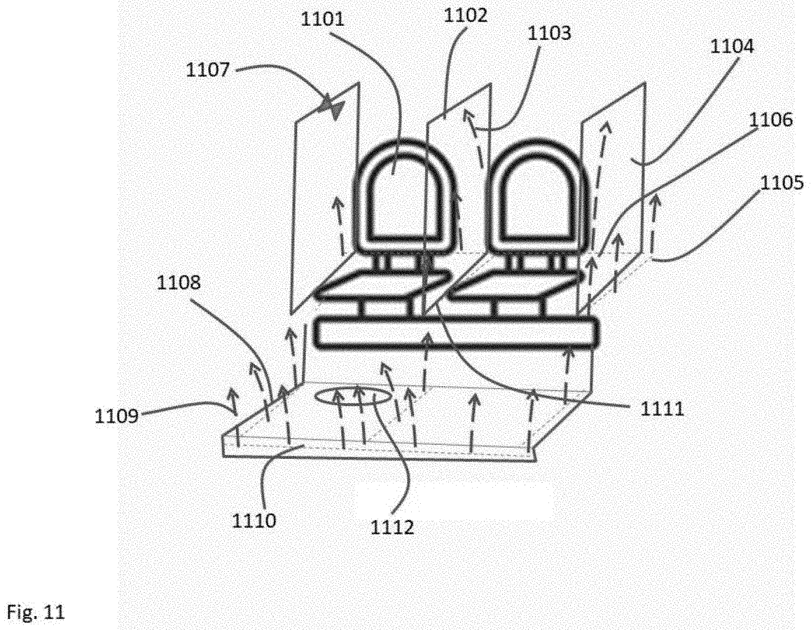

FIG. 11—Schematic diagram of a stadium seat pair:
1101—Seat;
1102—Middle screen;
1103—Hot gas and UV inside the screen;
1104—Lateral separator;
1105—Lateral hot air nozzle and UV screen;
1106—Back-seat barrier of hot-air and UV;
1107—Cold air nozzle;
1108—Floor hot air nozzle and UV;
1109—Ascendant hot-air;
1110—Hot air nozzle and UV emitter;
1111—Middle hot air nozzle;
1112—Cold air intake floor vent;

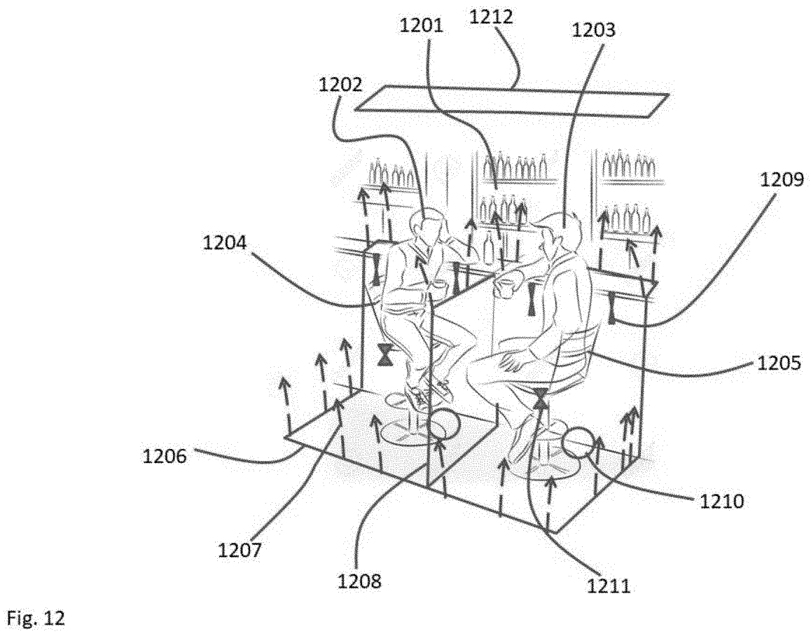

FIG. 12—bar seats improvement
1201—Bar shop;
1202—Left customer;
1203—Right customer;
1204—Left seat;
1205—Right seat;
1206—Floor surrounding hot-air nozzle and UV shield;
1207—Ascending hot air flow;
1208—Optional separator screen;
1209—Cold air blowing nozzle;
1210—Cold air floor intake vent;
1211—Under-seat Bernoulli effect nozzle;
1212—Celling hot air outtake vent;

### DETAILED DESCRIPTION OF THE INVENTION

The inventors consider the developments in siting and eating or drinking furniture for restaurants, cafeterias, bars stadiums, movie theaters, convention halls, decision boards, etc. where by default people needs to have both their mouth and nose circulating air in order to speak loud and clear or to eat or drink. These conditions are prominent zones for the spread of contagious diseases and present a risk factor for those around them. As such, air-box technologies and phototonic technologies can be used to create an almost invisible, seamless shielding to make such proximity safe.

In order to provide a safe pleasant encounter, the invention is using high levels of technology to increase the safety and to cancel any viral or bacterial transmission. It starts from real encounters, in restaurants and working cubicles where due to airborne droplets, transmission of COVID-19 can be spread easily, making people sick with a distribution and intensity that puzzled scientists.

The basics starts with the curves of airborne droplets emission by exhaling air from lungs in various regime, shown in FIG. 2, and their propagation range in open air, with understanding the properties of masks and their operation with liquid effluents, and aerodynamic variations.

It is known that the average adult inhales and exhales around 7 to 8 liters of air per minute when in a rested state. When coughing or shouting or exhaling air faster, watery droplets with dimensions up to 20 microns, are removed with air, with a maximum of the dimensional distribution of about 8 microns which may travel up to 2 meters in air, accompanied by submicron particles too. When breathing normally or speaking, a person releases mainly submicron watery particles, with a maximum at about 0.6 microns. These particles remain suspended in air for varying degrees of time depending on their size. It is about a week for 0.3 μm particles, to about 1 day for 0.6 μm, and about 2 h for 3 μm particles, being mainly airborne, while for 30 μm particles, it takes about 1 min. to fall 1 meter. After this time in the air, the particles are deposited and coat the surfaces underneath, or those which they come in contact with. Being airborne, these particulates are usually transported in ventilation systems, passing through AC systems and spreading through the entire building. When a sick person is inside, suppose being contagious with COVID, viruses released will be included in exhalation droplets. It is unclear how many contagious agents per droplet, because that depends on the contagiousness level. On average, an exhalation contains about 10,000 droplets/cc. When the AC system is set on cooling, the virus will concentrate in condensing water drain, and in part will pass unaffected, but when it is on hot, some of the viruses will be damaged by the heat, but not all of them, and the virus survival ratio will depend from case to case.

It is unclear how many droplet units and viruses units a person may intake to become sick, but from the previous data presented in FIG. 1 we may say that the person “A1”, charged the room with about 700 l of exhaled gas, containing 7 Billion droplets, which homogenized in the 100 m³ of the room and become a concentration of 70 droplets/cc, that inhaled for ½ h by other customers in total amount of 1.4 million droplets. In fact this data shows that a reduction of 5,000 times of droplets content gives enough safety for the process, setting the droplet retention rate at 99.95% as good enough.

Any mouth/nose covers helps in reducing the direct propagation range, but the passive airborne contamination is still present. In most cases, face cover protection is not applicable when intensive mouth usage is present such as eating, drinking, or speaking. The technologic system has to be designed to be aware of this restriction and manage it. In fact, we have to sterilize all released gases and stop them before they cross the center of the space, such as the table where the expectorated droplets and gases may have deposited. This must be done in an undetectable manner to not disturb the customers and give them the comfort they would have received if no equipment was present.

The main embodiments of the inventions are related to the process to stop water droplets from reaching nearby people, no matter their potential virus load, by using a vertical wind-shear in a system that resembles an atmospheric box. A cold air generator, AC system, is producing moderate cold air, say 20-28 C (68-82 F). Over it are water droplets/fog from a ultrasound humidifier, which is located over the customer's seat. The water droplets evaporate cooling the air even more, compensating for the customer's body heat and creating a virga-like effect. This falling air flow directs the customers ejected saliva and brings it to a cold air collector directly underneath them. The droplets produced from the customer's mouth and nose may be contagious, containing live bacteria and viruses that may be treated as a bio-hazard and a desiccator may be used to take them out of air, distillate the water to kill all bio-hazardous entities, and recirculated in an ultrasound atomizer. The collected air is warmed up to 200 C (392 F) applied in a catalytic burner felt, where all organic matter that passed through desiccator is burned out, and the air is released through the hot nozzles on the perimeter. This hot, very low relative humidity air will evaporate and warm up any droplet and make it lighter and float better. Then, after being collected by the hot air nozzle, reheated at 200 C (392 F) and passed again through a catalytic felt, where all organic compounds are burned and then pushed through a CO₂ arrester, then cooled down and reused in the cold air input.

Each customer is supposed to generate about 1 liter CO₂/min at maximum. Air may be released outside, via a heat exchanger, and new air from outside, UV sterilized and filtered through a HEPA filter which may be introduced in the AC cooling system in order to avoid oxygen concentration control in air recycling system. In order to save energy and have correct temperatures throughout, the heat will be transferred using heat-pipes and the temperature will be gently adjusted using electric heaters.

In order to save as much energy as possible, heat pumps and heat-pipes are used to move the heat with the airflow, and electric heat assistance is used to set the temperature at its optimum.

As lateral screens may assure enough privacy, the inside temperature may be varied, as to mimic a lunch on a hot beach, with associate tanning, sun simulation, scent simulation, an oxygen therapy session, even a sauna session; while enjoying food delivered by a robotic shuttle. Even further, one may use the booth to take a shower after lunch, or take a nap in a hammock if the sound surround is tuned on with the sound annihilation and sound and image on the desired theme. Other gases may be introduced, as secondary vaping, or nitrous oxides, except for the gases used for automatic sanitation and germs sterilization between the customers.

### 2. Best Mode of the Invention

FIG. 4 shows the devices in the best mode contemplated by the inventors where the customer is seated on a modular table fraction, forming a booth, possibly on wheels, that has all the technology working for a single person only. It is connected to instrumentation together with some solutions and developments that are embedded in the present invention.

There are very few models of modular booths, that may be connected in various ways forming many different configurations, as for example the 90 deg. boots (right triangle table) may form square tables for four people, rhomboid or triangle tables for two people, trapezoid tables for 3 people, rectangular tables or rectangular ring table for many people, and about the same may be achieved with an equilateral triangle table booth. Table presence is not a must for the booth, it may have the seat only or other inside accessories, as for sauna or sleeping.

Even more the two protection zones may be made mobile, and configured on spot, where it is possible to stack them together and make a protected space inside, making a mobile virus protected booth, or deck, where controlled atmosphere is present inside.

The invention was mainly conceived to assure airborne bio-threat protection for a seat-table assembly, in order to allow socializing in a safer manner for eating and drinking social customs, but it may well be applied to a sit only, or to a standup table; being suitable for meeting rooms, waiting rooms, stadiums, show halls, conference centers, etc.

The invention corrects previous deficiencies of the previous method, as follows:

It improves the safety of each person by separating the air spaces and destroying all organic matter by a combination of technologies and a redundant number of layers, which finally collects all fluid effluents emitted by a person and sterilizes them continuously maintaining the person in a fresh, safe environment, and cleaning even more in-depth the environment for the next person. In restaurant environments, human to human interactions are minimized by using electronic and computerized systems and robot systems for deliveries and cleanup.

As one can see in FIG. 7, the modularity is an important feature for maintenance point of view, each modular booth has to be connected to electricity, air intake and outtake ducts, water and sewage, for a full wide range operability, and of course to robotic food transport duct. If food delivery may be certified as safe, in a positive pressurized shuttle container, the return of dishes, is a risky operation, that will be performed only after customers departure, and will use a negative pressurized container.

Best application of the invention is explained in FIGS. 4 and 8, but it is not limited to the specific application presented and there are also some applications that do not require such complex equipment. A simplified version is possible to be used and gradually upgraded. It may start with versions that makes only seat surrounding protection using a curtain of UV light, but transition time of a virus and probability of damaging a virus by this method greatly remains unknown and protection failures are probable. The second stage in upgrading the structure is to apply a ultrasound nebulizer on top, in order to create a humid down current. But without extraction of the contaminated air, it will remain in the location, therefore one may like or not, it has to apply atmospheric box ventilation and take fresh air from outside, as fresh one may believe the air is outside and introduced by a fan over the nebulizer's water droplets, and warm the air on boundaries, make it flow up and flush it outside in the air. The problem is that temperature and humidity of air outside varies with weather, and one may need to control air parameters, therefore an AC unit introduction may be advised. The heat capacity is small, of about 100 BTU or less and at a heating efficiency of 10 and SEER 16-20 it turns to use about 100 W or less to do the job. Up to now, one assured the customer's comfort, but still has to deal with bio-threat from effluents. In order to make it safe, one has to destroy the effluents, but it must first be collected or the entire mass of air involved in the process must be sterilized. Collection in desiccators and/or scrubbers is useful, sometimes unavoidable when trying to use same air in cold and hot barriers.

In hot barriers, air must not be heated so high as it becomes dangerous in people's proximity, producing burns. This must be avoided but therefore, it is uncertain how efficient the heater only is in destroying viruses and bacteria, and a complete solution might be required.

We arrived, step by step, to the solution presented in FIG. 7 where we are adding some comfort features, as electronic communication, robotic shuttle food delivery, robotic cleanup, privacy screens, with virtual environment, sealed atmosphere control, which at some point may become dangerous, as allows opens the possibility of gas selection by user.

### 3. How to Make the Invention

As one can see from the drawings in FIG. 8 this method and procedure includes a device that is conceived to prevent the most complex accidents actually known by accidental sneezing or coughing, being usable in any other activity as meeting or stadium safe and on its optimum.

First one has to make the basic structure of the booth, and decide it is a restaurant eating module, what is the prism's angle (60, 90), or a stadium, theater or conference seat, and find the right materials to build it. There are preferred UV resistant materials as first approximation, no fluffy stuff, water resistant, and coated with active bio-sterile surfaces, that might be easy to sterilize.

After the structure contains double deck at bottom and a double celling, the bottom is made as a shower swamp, watertight, with a single water drain easy to connect to infrastructure by a specialized hose. Under the upper deck, the technologic devices will be placed, in such a manner as to be vibration and noise free and to be able to operate underwater, as water proof, in case of an accident, with massive liquid spilling.

With exception for fans, all heat is to be commanded from above using heat-pipes as minimal electric power to be added underneath and this is to be at low voltage under 24 V.

The boundary line has to contain hot air curtains that are controlled in selective manner, as first activated the front curtains and then the back curtains on the boundary. UV may help increase sterilization, but it also exhibits some risks for people looking directly into the beam, and with all protection being necessary to be an open source brings a hazard, therefore a supplementary optical barrier, acting in front of hot air curtain has to be used to prevent any penetration, and prevent burns from hot air or UV light.

In the center of the space, the cold air descending flow is placed, where speed is given by the fans in celling and floor, and is reciprocated by the hot air fans in floor and ceiling, therefore the input flow has to be equal with the output flow and pressure maintained constant. For safety reasons, one may like to get a few mm of water negative pressure there, but that is possible only for closed, air tight booths assemblies.

For energy economy, it is recommended to use a small AC unit but a multi-split unit may work as well, and integrate them in the air ducts. An ultrasound nebulizer might be useful to create water droplets to release cold air from the AC vent, which will interact with the air droplets exhaled, increasing their dimensions and make them condensate on a cold source or in a desiccator, removing in one step more than 90% of hazardous bio-agents exhaled. Another option is to directly warm up at 200 C the air with droplets now becoming vapors, in the hope as viruses and bacteria will be killed, and sent upwards directly, because such a stream has higher mass and specific heat, than air, going faster up, but is also increasing the burns hazard. This will be a very simplified construction, because after the floor fan, the air will be warmed up directly using a heat-pipe and sent in the hot air nozzles.

Some electronics, a tablet and sound system, have to be integrated near-by for communication purposes. All the time there will be issues with respect to confidentiality, and people will avoid going to eat and talking sensitive issues on contracts, collaborations with hidden selection criteria, due to confidentiality issues unrisen by technologic superiority.

For cases that want enjoy the moment, a sound-surround noise suppression system may be added, and screens around with preferred themes. Most of the technology will be set above ceiling, and a clean air in with clean air out principle will be applied, therefore all organic matter coming in or out will be burned in catalytic devices, and input air will be filtered by dust and depleted of CO₂ and other nasty gases. In some cases, N₂O/O₂ 50/50% may be added, or scents, or even with full privacy a sauna, or tanning bath, or shower a customer may take.

For the restaurant version in prism booth, a space has to be provided for a sealed robotic shuttle, to carry in a sterilized manner, with positive pressure and UV the foods, and deliver, as well to carry back the dishes, sealed in a negative pressure and UV atmosphere, using different ducts, and only the booth has a common duct, coming from above the booth.

As a supplementary feature, it is possible to add a shock wave detection system, which will trigger compressed air jets that will intercept the jet exhaled by the customer's mouth, stopping it inside the customer's airspace. Care has to be taken, when realizing these jets that to produce a constructive interference in a space at least 20 cm away from customer's face.

As constructive solution, the level of protection may vary, in agreement with customer's desire, from UV curtain, preferably UV-C that has a small ozone production that is supposed to kill the viruses or germs crossing the curtain, but one has to pay attention to undesired reflections, that may be harmful. Next level is air flow curtain protection, that collects the exhaled air, and drives in sterilization units using moderate heat, up to 200 C (392 F) and catalytic incinerator methods or collection by condensation and boiling, such as the exhaust air to be free of any organic matter. As an extension, these protective measures may be applied in hospitals too, but with care considering the specifics of each activity. In tall halls, the upper floor over ceiling may host some sliding screens that once in place become air tight, surrounding a group of booths that forms a table, assuring an advanced privacy, and enhanced atmosphere, gas therapy, etc.

The purpose of the patent is on how we can restore normality by using protective technology in the presence of bio-threats on human unidentified carriers. In fact, in conditions of a permanent threat from COVID-19, we want to make possible to have parties like that in FIG. 3, without the consequences described in FIG. 1, by using technology to eliminate the mutual spraying with saliva and other liquids and bacteria from lungs, and by this way to stop sharing of any bio-agents, and keep the participants health status unchanged by the event. It is clear for us that simple “social distancing” and “masks” is not enough to make restaurants and bars profitable and safe again, and only these types of additions may solve the problem. We intend to make sports arenas, conference rooms, and theatres populated again and for everybody to enjoy proximity and stay safe. As we stated, it is possible that instead of tables, a hammock or a hospital bed may be placed to keep airspaces separated from bio-agents, or use the technology for keeping air clean in various enclosures.

### DETAILED DESCRIPTION OF THE FIGURES

The technology is intended to prevent the spread of COVID-19 and other viruses while also being comfortable and almost unnoticeable for users. To allow this, a sophisticated air flow control system as well as other virus propagation suppressors and clean air generation is required.

FIG. 1 shows more details using a schematic view of the restaurant X, incident showing arrangement of restaurant tables and air conditioning airflow at site of outbreak of 2019 novel coronavirus disease, Guangzhou, China, 2020, January 23;

This is a real story that triggered our attention because on Jan. 23, 2020, a Chinese family “A” traveled from Wuhan and arrived in Guangzhou. “On January 24, the index case-patient (patient “A1”, 112) ate lunch with 4 other family members (“A2”-“A5”, 123, 127, 126, 131) at restaurant X. Two other families, “B” and “C”, sat at neighboring tables, 116, 117, at the same restaurant. Later that day, patient “A1”, 112, experienced onset of fever and cough and went to the hospital. By February 5, a total of 9 others (4 members of family “A”, 3 members of family “B”, and 2 members of family “C”) had become ill with COVID-19.

Based on coordinate cell-phone back-tracking the only known source of exposure for the affected persons in families “B” and “C” was patient “A1”, 112, at the restaurant. It was determined that virus had been transmitted to more than 1 member of family “B” and more than 1 member of family “C” at the restaurant and that further infections in families “B” and “C” resulted from within-family transmission.

The restaurant was placed in a 5 floor building, having restaurant hall walls, 101, on the SW side of the building, and it is an air-conditioned, 5-floor building without windows. The third floor dining area occupies 145 m²; each floor has its own air conditioner 103, 104. The distance between each table, 117, 115, 125, is about 1 m. Families “A” and “B” were each seated for an overlapping period of 53 minutes and families “A” and “C” for an overlapping period of 73 minutes. The air outlet and the return air inlet for the central air conditioner, 103, 113, were located above table C, with a return 104, 114, above table B.

On January 24, a total of 91 persons (83 customers, 8 staff members) were in the restaurant. Of these, a total of 83 had eaten lunch at 15 tables on the third floor. Among the 83 customers, 10 became ill with COVID-19; the other 73 were identified as close contacts and quarantined for 14 days. During that period, no symptoms developed, and throat swab samples from the contacts and 6 smear samples from the air conditioner (3 from the air outlet and 3 from the air inlet) were negative for severe acute respiratory syndrome coronavirus 2 by reverse transcription.

From specialist's examination of the potential routes of transmission, they concluded that the most likely cause of this outbreak was droplet transmission. Although the index patient (patient “A1”, 112) was asymptomatic during the lunch, pre-symptomatic transmission has been reported. Given the incubation periods for family “B”, the most likely scenario is that all 3 family “B” members were directly infected by patient “A1”, 112. However, we cannot not exclude the possibility that patients “B2”, 129, and “B3”, 130, were infected by patient “B1”, 128, afterwards, the first family “B” member to become ill. For family “C”, a possible scenario is that both patients “C1”, 126, and “C2”, 124, were infected by patient “A1”, 112; another scenario is that the patient “C1”, 126, acquired the infection while caring for patient “C2”, 124, beginning on January 27.

At that time, the authors considered that virus transmission in this outbreak cannot be explained by droplet transmission alone. Larger respiratory droplets (>5 μm) remain in the air for only a short time and travel only short distances, generally <1 m. The distances between patient “A1”, 112, and persons at other tables, especially those at table “C”, were all >1 m. However, strong airflow from the air conditioner could have propagated droplets from table “C” to table “A”, then to table “B”, and then back to table “C”. In order to clarify this aspect we included FIG. 2.

Virus-laden small (<5 μm) aerosolized droplets can remain in the air and travel long distances, >1 m, in the case of potential aerosol transmission of severe acute respiratory syndrome and Middle East respiratory syndrome viruses has been reported. However, none of the staff or other diners in restaurant X were infected. Moreover, the smear samples from the air conditioner were all nucleotide negative. This finding is less consistent with aerosol transmission. However, aerosols would tend to follow the airflow, and the lower concentrations of aerosols at greater distances might have been insufficient to cause infection in other parts of the restaurant.

WE further analyzed the schematics in the restaurant interior 3 table row on west side, 102, that had a north wall air conditioning unit, 103, 113, in zoomed sketch, and a south wall air conditioning units; 104, respectively 114, producing direct air flow lines from N to S, 105, and in reverse air flow from S to N inside the room, 106.

In the same hall, there was a second row of tables, 107, that were unaffected, and the area of interest where COV transmission occurred, 108, was selected for further analysis while the other half of restaurant hall, 109, where was no transmission because it had separated air flow currents, 110, inside the second half of restaurant hall.

The area of interest in a detailed diagram, 111, shows that an infected, contagious person “A1”, 112, who was declared sick, next day on January 24, occupied a seat in the middle of the line, where on N wall is an AC unit, 113, and a similar one is on S wall, an AC unit, 114.

At a central table “A”, 115, on W side of the hall, near it had to SW table “B”, 116, and to NW table “C”, 117. Towards E was central row of tables, 118, nearby, “E” and “F”, where un-contaminated people on seats, 119, were placed.

In order to highlight on complexity of the problem, we highlighted client's “A1” exhaust plume just exhaled, 120, with a volume of about 1 gal. of air infested with micron and submicron size watery droplets, some containing the COV too, and at a little distance another one, previously exhaled aerosol droplets contaminated plume, 121, a little larger due to dispersion, and another ones previously exhaled plume, driven by air currents at table “C”, 122, and so on, plumes trained in the room by the air currents, until the virus is completely homogenized, and meanwhile it dies or is removed via condenser.

The chain of infections is very intricate, because we only know the positions at table and the time they were fallen sick, and we suppose that the delay in falling sick to be determined by the amount of virus initially inhaled, but other unknown competitive processes have been also presented before. What we know is that family's “A” member “A2”, 123, was infected and found positive in 4 days; sitting diagonal on the table, 4 ft. away, in the direct range of plumes exhaled by “A1”, and on about the same direction was family's “C” member “C2”, 124, who was infected and found positive in 4 days, on January 27, siting at about 10 ft. away, but possible in the AC air currents 105.

Family's “C” member “C1”, 125, was also infected and found positive in 8 days, on January 31, two days after family's “A” member, “A4”, 126, infected and found positive in 6 days, on January 29, about same time with family's “A” member “A3” 127 infected and found positive in 6 days, on January 29. At the nearby table family's “B” member “B1”, 128, was infected and found positive after 9 days, on February 1, while family's “B” member “B2”, 129, was infected and positive after 13 days, on February 5^{th}, by the same time with family's “B” member “B3”, 130, infected and positive after 13 days on February 5^{th}, without having no good explanation on why other persons around din not fall sick.

We conclude that in this outbreak, droplet transmission was prompted by air-conditioned ventilation. The key factor for infection was the direction of the airflow. Of note, patient “B3”, 130, was afebrile and 1% of the patients in this outbreak were asymptomatic, providing a potential source of outbreaks among the public. To prevent spread of COVID-19 in restaurants, the authors of this study recommended strengthening temperature-monitoring surveillance, increasing the distance between tables, and improving ventilation, that in our opinion is not enough, leaving the possibility open that other customers to inhale into contaminated plumes.”

FIG. 2 shows a chart of exhaled water droplet distribution, vs. filter retention capabilities, as reference to COVID-19 average dimension of 100 nm, 201, with a reasonable droplet size scale, 202, ranging from 0.1-100 microns. On left ordinate axis is represented the number of particles per cc (cubic centimeters) as function of particle magnitude, 203, and also particle filter 100% pass upper limit, 204, is conveniently placed, on top, while particle filter 0% pass=100% stopping power, lower limit, 205, is placed on the opposite side. Time airborne droplets float in air scale, 206, where the droplets have 1.5 g/cc density, is there to show how long a certain type of droplets may be resident in a closed room atmosphere and may accumulate. In the upper right corner it is presented the case of a person that exhales and sets in air droplets that may or may not contain viruses inside, where their concentration is given by a color code, 207, and applies for the virus inside an air exhalation too. Exhaled air-jet change of direction due to mouth cover, 208, and makes it turn downwards, while exhalation plume without any face cover, 209, may go as far as 8 ft (2.4 m), and is also influenced by wind's direction, 210, in correlation with the orientation of the head of a contagious person, 211.

In order to understand how a protective measure works, one must first understand the dimensional distribution of airborne droplets while coughing openly, 212, then what happens when coughing is performed with retention in a new N95 mask, first shout, 213, where conversion of large droplets into small droplets released by mask by atomization, 214, takes place, making the things more complex due to extending the airborne time of droplets, with increased accumulation. Dimensional distribution of droplets released through a paper-towel filter, 215, is a little bit larger, but atomization process is not as strong and the release of droplets spectrum through is smaller than that for a N95 mask (without exhaust valve); 216. But overall, the results are about the same. In order to understand the uselessness of a N95 mask or filter, one has to consider the dimensional distribution of droplets during an open exhalation, 218, versus normal breathing and air exhalation through a mask, 217 and see that at best, when new it is a 50% reduction only, while the liquid effluents accumulated in the mask are further released, in the dressing/undressing room and storage room where most of contaminations take place.

The airborne time was approximated with the time it takes a droplet to free-fall 1 m (3.3 ft) based on Stokes force, represented by the solid line, 219, and over for clarification is given in percent the paper towel filter retention curve, 221, and N95 filter retention curve, 222, where one may see that none of them really stops under micron particles, nor liquid effluents.

Applying this information to the case presented in FIG. 1, one may see that for customer “, A1”, 112, sitting 90 min, about 0.45-0.6 m³ of infested air is released, with COVID-19 viruses floating in water droplets and accumulating, gradually spreading and mixing in the entire building, first inside the W half having 6×8×2.5 m giving 120 m³, where each cubic centimeter of exhausted gas contains about 10,000 droplets, giving a final concentration in the room of about 50 contaminated droplets per cubic centimeter, with a dilution factor of 200×. What was hard to consider was the action of AC system, that if set on cooling condensates the air, removing fluid effluents into condense drain, or if it is cold, it heats the air over 80 C locally evaporating the droplets, and killing them, and that in part explains why only 10% of people there got sick.

FIG. 3 shows the actual state of the art with respect to parties and socializing eating sessions, around a large table, 300, where an effervescent speaker, 301, is shouting, covering all the seats on his right with a plume of droplets, 302, exhaled during effervescent speech, with a dimensional distribution looking similar to a cough. At the other end of the table another arguing speaker, 303, is caught in the debate and shoots his arguing speaker's plume, 304, up to a person listening, 305, over a person arguing over the table, 306, who at his turn, shoots his own plume, 307, over the table, covering both the person listening, 308, near him, and a person eating, 309, at the left of the person arguing back, 310, over the table. At the other end of the table is a person amazed, 311, by parallel talks, another person is bored, 312, another person is ecstatic, 313, and an old lady, 314, is listening. Finally, from an epidemiologic point of view, almost all the exhausted mouth droplets are floating in the air over the table and guests for more than 5 min., giving everyone enough opportunities to inhale a significant amount of droplets from everybody, and in short time if one is contagious almost everybody might be infected. This is because in the absence of ventilation, the air is almost static, floating around for days, and even the people in charge with cleanup after party may get infected.

FIG. 4 shows details on a table module that is the elementary part, a main embodiment of the present invention that deals with 1 customer, or a customer with a little child, if made larger. In this kind of booth, a customer, 401, is sitting at a table section that may be ¼ or ⅙ of a full classical table, but may also be grouped with a spacer, 431, for multiple purposes, as customer, waiter access, robotic waiter, or other grouping arrangements. Customer is placed on a simple, UV resistant, washable ergonomic chair, 402, adjustable for having best comfort holding the hands or eating on the table top, 403, made of a material that may be coated with paint containing active nano-particles as silver oxide, rutile, etc., that kills bacteria and viruses by direct chemical interaction or light activated. The surface has to be easy washable, and able to hold disposable septic table cover.

A table lateral protective structure, 404, that has linear hot air nozzle and UV retro-reflector all along the perimeter, such as it creates a sterile virtual wall made of hot gas and UV-C light, that is invisible for the customer.

A hot air ascendant, lamellar jet, 405, makes the lateral surfaces aerosol sealing of customer, such as water droplets exhausted by mouth, which are first driven downwards by the cold descendent air, 419, but if it escapes from there, they enter in hot air ascendant jet, 405, that dries them, and damages by UV irradiation, being trailed upwards into hot air absorption holes, 406, mounted inside double layer celling, 407, containing collection tubes, that drives air towards hot air exhaust, 408, from where it is safely dumped into the atmosphere, or treated and recirculated.

Fresh intake airflow, 409, takes air from outside, or from the air reprocessing unit, and brings it to local input of air processing unit that contains a dust filter, dehumidifier, temperature setting, air composition setting, where various scents may be introduced, 410, and which exchanges heat flow via a heat-pipe assembly, 411, and heat-pump, such as the energy consumption to be minimized.

The customer emitted aerosol containment is achieved using a border region hot air laminar flow; 412, going up, in a shearing current with descendent cold air vortex coming from a celling hole and vent, 415, that brings down the air produced by the air cooler unit, 414. It is desired that all customer belongings to stay with customer, therefore an internal wall for dress, coat hanger, 416, is provided being washed by dry warm air jet, 417, for coat decontamination.

A descendent air-flow over customer, 418, and a descendent airflow in front of customer, 419, makes that all aerosols and droplets released by customer to be contained and collected in a used cold air intake, 422, via used cold air ducts, 424, inside walls and fake floor, and driven to a heat exchanger and catalytic burner hot air cleaner, 421, that burns on a catalyst all organic fractions transforming into carbon dioxide. Following this, it goes through hot air flow internal tubing, 420, to hot air exit ducts and nozzles, 423. To assure consistency of sharing wind, covering the customer, 401, a central, under-chair used cold-air floor intake, 425, is used, to collect all cold air, without creating differential pressure that to perturb the liminality and stability of the air flow, that resembles a convection pattern.

Hot air perimeter nozzles and UV corner-back-reflector, 426, placed on lower floor plate; 427 are providing full protection all around. Between customers, or even at customer request transparent, or opaque or video screen separator; 428, it may be lowered between the tables, sealing them air tight such as a special atmosphere may be induced, a privacy system with lights and screen images, or in customer absence, sterilizing gas and UV may be applied to the entire enclosure, not only on border UV chain LEDs, 429.

Tip angle or profile, 430, is determining if the module may serve into a hexagonal, triangle, octagonal, or square table assembly, or be mounted in rectangular structures.

Unexpected incidents as coughing or shouting may happen, and microphone/loudspeaker pulsed phased array, 433, is detecting the sound, and generates an air-pressure pulse, to meet the pressure wave and cancel in the boundary between descending cold air and ascending hot air. This action will be enforced by an action from compressed air puff generator, 431, in the cold air section of the person producing the incident, and in the subsequent sections where a hot compressed air puff generator, 432, is triggered in both sides, with the aim of stopping large spit particles exhausted at few m/s speed, and with equivalent Stokes speeds under 1 m/s, being about 1 mm in diameter. The combination between the sound pulse wave and longer pressure pulse will stop and deflect the exhaust. If spit or aggression is detected, an N₂O gas, used for anesthesia will be pulsed over aggressor, and alarm will be triggered, unit security team will be called to evacuate the table or seats. Light, sound and filming will be performed to prove the quality assurance during the evacuation process.

A lateral board, 431, may be used as a variant and for access purposes, while putting the modules on wheels, the restaurant hall reconfiguration is easy and multifunctional.

FIG. 5 describes the a cubic table for a four person system, assembled from modular units with tip angle of 90 deg. that forms a four seats equivalent table, 501, with diagonal hot air and UV barrier, surrounded by chairs, 502, each inside a module, where the customer is protected against aerial droplet exchange by diagonal separation zone, 503, and lateral border zone, 504, containing hot air laminar nozzles and UV forming a double protection curtain.

Bottom floor layer, 505, may be on wheels or with fork-lift adaptors, for easy reconfiguration and contains all flor technologic equipment that handles the customer released gases, clean them and send up as hot jet, towards celling layer, 506, all along the module borders, including diagonal separation and protection zone, 507, very important for lateral and front customer protection.

Lateral upper celling zone, 508, contains sliding mechanisms for sliding lateral separation screen, 509, with multiple function, that may completely separate the triangular-prism module when is using diagonal, sliding separation screen, 510, to make an air tight room, for washing with shower liquids, hot or chemical active gases, during decontamination process, to offer the customer a private enclosure with images projected on screen or light curtain, or a sauna experience, or most often, a pleasant fresh air restaurant experience.

Above each module there is a fresh air input duct, 511, bringing air from outside or from a respiratory gas synthesis unit that may recycle internal air or take fresh air from outside, filter, decontaminate, and bring as fresh air. A hot air output duct, 512, is used to collect all air exhausted by modules and introduce into a reprocessing unit, extracting the excess energy, and flush outside or into recirculation process.

In center of the table assembly a robot duct is set, to allow access of a robotic transport device, a shuttle, carrying food for customers, forming a food delivery robotic access duct, 514, continued on vertical of the center of the table with a vertical access sliding duct, 515, where the shuttle is descending at table level, opens and delivers the food according to customer's order, where the table protection limit and central access assembly, 516, plays an important role in redundant airborne droplet exchange prevention. When the robot's tray with a plate with food on it crosses over the hot jet the protection is compromised, but on the other side, the protection is functional, and the aerosols leakage from one customer space into another is prevented.

Protection may be increased, for the case the separation screen are up, by using a redundant protection formed of a first ring of protection, 517, just behind the customer's chair, and the lateral and diagonal protection curtains, covering also vertical pillars structure support, 518, that together makes a very stiff structure.

FIG. 6 gives details on a hexagonal table equivalent, made of 60 deg. module booths with round border, 601, or module booth with table having a linear edge, 602, each having a stylish or ergonomic chair, 603.

Because the table has to be sterilized, ornamental plants, 605, or other ornamental objects that are difficult to sterilize are not present inside.

Upper ceiling round cover, 607, contains UV LEDs and hot gas intake nozzles, where the structure is holding on a lateral pillar, 608, and it also contains the hexagonal boundary, upper and a Round lower floor, 610, and round upper floor with hot air and UV shield, 611.

When a dish is placed in wrong position, 612, over table top separator and hot air, UV screen, 615, that continues on the table the lateral separator and hot air, UV screen, 614, a photocell or camera will detect that, sound an alarm, illuminate red the object, while stopping the hot air exhaust, signaling that safety have been compromised in part. Same happens when a chair placed in wrong position, 617. On the central table may be placed a dish or distribution robot, 616, communicating via top or bottom with the kitchen for food and drink delivery.

FIG. 7 describes a restaurant floor example made of 8 hexagonal structures connected inside a hall, similar to a bunch of pergolas, installed one near another inside a tall restaurant hall, 701, each table being made of 6 table module as a 60 deg. triangular prism booth on first row, 702, and similar table module as a 60 deg. triangular prism booth on second row, 703.

A customer on 2; 1; 1 seat, 704, may enjoy the presence of up to 6 friends, at the 2.1 table, being separated from each other by invisible seamless hot air and UV shields, and upon request by solid screens, that may become mate, or project images, so they may be separated even more.

A hexagonal boundary with protective shields, 705, may be lowered, containing the invisible hot-gas and UV shields, and may be acted even in the joint among structures, 706.

Central table spot, 707, may be used for robotic food and drinks delivery, or simply holding a decorative object most likely a hologram, where a transport robot may use a robot access duct, 708, to safely deliver the food and drinks to customers, in a sterile manner, where the transport vehicle may be equipped with UV sterilization lights, and pressurized enclosure.

FIG. 8 describes some of the main embodiments of the inventions that are related to the process to stop water droplets from reaching the nearby persons, no matter their potential virus load, by using a vertical wind shear in a system that resembles an atmospheric box, and describes the air flow and protective assemblies inside the seat-booth. Usually the air is collected from outside in the case it is clean outside, or from inside in case outside air is highly contaminated, or a mixture with partial air recirculation. Most commonly, the “atmospheric box” where the customer is seated, 801, is designed to annihilate any bio-chemical hazard exhausting customer's head, 802, considered as main source of hazardous airborne effluents, but all customer's body emissions are treated and passivated. In most cases, except for “shelter in place situations”, when outside air becomes hazardous, air intake is done from atmosphere, 803, through a duct to an air intake filtering system and HEPA filter, 805, from where air output in gas/gas heat exchanger, 806, getting the right temperature to enter a catalytic organic matter burner and CO₂ sequester, 807, that burns all viruses, bacteria, hydrocarbons, etc., and also takes air from recirculating air optional input, 808, where air reprocessed from inside comes and is recirculated or sent as hot air exhaust, 809, after being passed through a hot air filter and catalytic burner, 810, in order to be compliant with environmental standards. A hot air intake, 811, is collecting the air coming out via an AC unit, cold side, 814, to recover the heat inside the system, in spite of heterogeneity in temperature field that was created. For energy saving purposes a heat pipe, 812, is used to recover the heat between the input and output of the catalytic burner.

Cold air exit from carbon dioxide arrester/sequester and input into AC unit cold side, 815, drives the air flow into an AC unit cooler, 816, where recycled water condenser, 817, is recycling water in the process and adds it into an ultrasound atomizer/nebulizer, 818, that makes water droplets insertion in AV vent in a celling cold air vent, where a down coming airflow, 819, is splitting around the customer, washing it downwards, all-around. Some airflow fringes may be trained in air vortexes, 820, interacting with hot air up-flow coming from hot air boundary nozzle, 821, that forms secondary air flow curtain.

The most unpleasant incidents are sudden sneezing, coughing, and a complex audio system, containing a microphone loud speaker system for multipurpose action, 841, detects the incident and in real time triggers a cold air puff to stop sneezing exhaled jet; 822, from a direction and another cold air puff to stop sneezing exhaled jet, 825, from another direction, which meet in front of sneezing water droplets penetration through down going cold airflow, 824, and creates by constructive interference anti-sneeze puff, 826, that slows down the airborne droplets, sending them back into down coming cold airflow, 823, possibly creating a down on floor or table top vortex, 827, that pushes the sneeze or exhalation airborne droplets plume, 828, into down going cold airflow, 829, and further outgoing cold airflow through floor vent, 830.

Due to this unexpected incident, or during normal activity, we collected a lot of airborne droplets some of them representing a bio-hazard, and we have to deal with them by making airflow enter the air cooler-desiccator unit, 831, where viruses in air droplets are removed with condensate water, from a cooler-desiccator unit followed by air heater, 832, where water condensate in drain is driven to vaporizer, 833, where bacteria is killed by temperature, and a vaporizer water transfer pipe, 834, drives vapors to recycled water condenser, 817. It is still possible to have gas effluents that were not passivated by desiccator, therefore heated air enters the catalytic burner, 835, where all organic compounds in catalytic burner, 836, are reduced to carbon dioxide, other oxides and water, therefore hot air exit from catalytic burner passes through boundary nozzles, 837, and through floor boundary nozzles for forming a hot airflow curtain, 838.

A boundary retractable multi-purpose screen, 839, is sealing the booth for sterilization purposes between different customers, and some may be equipped with LCD screens and place various images, changing the ambient and increasing privacy.

A chain of UV-C sterilizing lamps form a curtain reflected on corner reflectors surface, 840, and is a supplementary barrier, while the paint on surface is photo-active, containing Pt, Pd, Ti, Ni, AgO nano-particles with septic effect.

At the customer's request, an oxygen therapy may be also applied, by introducing an atmosphere with increased oxygen content, or using Cl gas to sterilize the booth, by using multi gas tubes for multipurpose action, 842.

Seat-table modular booth assembly, 843, level technology is desired for easier maintenance. Large flexibility of services that may be provided inside the same hall by simply changing configurations and taking advantage of large modularity.

FIG. 9 shows a section through a customer sitting at a cafeteria table, as an application example, and embodiment of the present invention where a customer, 901, sitting at a table, 902, on a chair, 903, is ventilated from above with cold air, containing water vapors, produced by a humidifier, and applied by an upper cold air vent, 904, that is surrounded by an upper-central hot air absorption nozzle array, 905, which collects the ascending hot air, 906, and the upper-lateral ascending hot air in an absorption nozzle array, 907, that formed a lateral hot air shield ascending current, 908, reinforced by an UV shield, 909.

Descending cold air with aerosol produced by an ultra-sound humidifier, or equivalent, 910, forms a kind of virga-rain effect, creating a rotating descendent air that coats the customer, and is absorbed by a cold air bottom intake vent, 911, which works in corroboration with the perimeter hot-air and UV shield, 912, and central hot-air and UV shield, 914, all the flow looking like a pill-box convection, where the inner cold current collects almost all droplets exhausted by customer, drives them in a burner under the floor and returns as surrounding hot air that prevents any outside droplets from reaching the customer, and the escaped droplets from the customer, with larger particle magnitude are collected by hot air ascendant flow, dried and evaporated and took out as an exhaust or return into an air processing equipment recirculated as cold air again, preferably over the same customer. Opposite chair, 915, may trigger or may not trigger central table ascending hot-air and UV shield, 916, activation, all around including lateral, back of opposite chair hot-air and UV shield, 917. The final goal are the customers, sick or not to be prevented of sharing their viruses while inside the restaurant or sitting at dinner table. In spite we can, there is not our goal to detect their bio-medical parameters in order to vary the attitude towards them.

FIG. 10 describes an application of the airborne droplets shielding in the actual restaurant environment, where a woman is sitting at a table, 1001, having a coffee on table top, 1002, and sitting on a chair on left, 1003, chatting with man, 1011, sitting at table, 1002, sitting on a chair on the right, 1014, face to face at a distance smaller than 5 ft (1.5 m). In normal condition, spit coming out from their mouths as micron size airborne droplets, will reach 6-7 ft (about 2 m) range, and both will spray each other, in an action they might call a nice, useful talk. In the case of pandemic, this is dangerous, because for people with different histories of virus exposure, the transmission of virus is undesirable, therefore in order to enjoy the talk we had to use seamless technology to suppress spit spraying.

This is done using a cold air upper vent, 1004, that releases a cold air down-current, 1005, which after washing the customer, is collected in a cold air intake vent, 1008, in a fake floor, that generates a hot air ascendant current, 1006, all around on perimeter, using hot air nozzle, 1007, that extends under table by an additional vertical separator, 1009, that physically separates their legs spaces, in continuation of a separator floor hot-air nozzle, 1010, that surrounds the cold-air intake vent, 1012. This kind of booth for two, may have a separation screen, 1015, enhanced by a supplementary privacy screen, 1024, equipped with a hot air nozzle and UV shield at back screen, 1025.

In these conditions the woman's exhaled gas plume trained downwards by cold air flow, 1016, while a fraction of woman's exhale plume trained upwards by hot air flow, 1017, and coffee's vapors trained upwards and vortexing, 1018, in the gas exhausted by table-top hot-air nozzle and UV screen, 1019. Even if her spit particles penetrate the two air shields, they pass through UV light that sterilizes them and enters in the next barrier of her partner that carries them upwards.

Man's exhale plume trained downwards by cold air flow, 1021, by descending cold air flow 1022, exiting cold air upper vent, 1023, and a little bit of the man's exhaled plume is brought upwards by hot air flow, 1020, may be crossing in the woman's hot air and UV shield, and being removed into a general air reprocessing system.

FIG. 11 shows a schematic diagram of a stadium seat pair, where a seat, 1101, is separated by a middle screen, 1102, equipped with a hot gas flow and UV inside the screen, 1103, and a lateral separator, 1104, equipped with an array of lateral hot air nozzle and UV screen, 1105. A back-seat barrier of hot-air and UV, 1106, improves the back safety, while a cold air nozzle, 1107, shoots over the spectator a cold, humid air that is collected by a cold air intake floor vent, 1112.

A floor embedded hot air nozzle and UV, 1108, releases an ascendant hot-air, 1109, released from a hot air nozzle and UV emitter; 1110, same as middle hot air nozzle array, 1111, which produces an antiseptic seamless screen, protecting spectators against any exhaling droplets.

FIG. 12 shows a bar seats improvement, where in a bar shop, 1201, a left customer, 1202, seated on left seat, 1204, enjoys drinking something with the right customer, 1203, seated on right seat, 1205. A floor surrounding hot-air nozzle and UV shield, 1206, produces an ascending hot air flow, 1207, that shields the consumers. More, an optional separator screen, 1208, may be inserted between consumers to increase safety. A cold air blowing nozzle set, 1209, and one placed under-seat, producing a Bernoulli effect, at nozzle, 1211, is pushing cold air floor intake vent; 1210, while hot air is travelling in opposite direction towards outtake vent; 1212.

### EXAMPLES OF THE INVENTION

The current idea was to facilitate a reduction in social distancing, which alone is not enough to assure safety of people involved. By increasing the safety, no matter if the participants are contagious or not, while on premises using the special tables or booths no virus or bacteria interchange occurs, people may enjoy the proximity of others without any visible separators. Of course, touching through air and UV barriers will be not recommended, and a system will detect that compromise of safety and ring the alarm.

The present patent is not intended to drastically change how parties and interactions are performed, but compared to actual parties where everybody in that room is sprayed with the spit droplets of everybody there, and is breathing micron size watery droplets exhaled, luncheons and other eating and drinking socializing activities will take place in a sterile environment where the participants exhaled air is first sanitized, purified and then recycled, or exhausted and breathing air will be provided from fresh, clean outside air.

When the present invention is applied to a participant or a group of restaurant customers, the customers take places on seats placed around a table designated for the number of occupancy, as 1; 2; 3; 4, . . . and more, which were previously sanitized, and the air and UV shields are activated.

The order will be done on computerized displays, and a robotic shuttle will deliver the foods and drinks, in safe and sterile conditions, under quality assurance. The only system allowed to cross the UV and hot-air barriers will be the robotic arms transferring the foods to customer.

When successful, this technology will eliminate the bacterial exchange that exists in actual restaurants, bars, airports, stadiums and other public places, where people have to sit and wait.

A sit or a sit-table assembly has an operation cycle that encounter following steps:
Preparation to receive a customer—that mainly produces decontamination and sterilization of the seat-table booth assembly. As a design feature they have only round, easy accessible places, visible directly for UV light, and easy to wash, without nook or crevices between connections of an assembly, where dirt and germs may accumulate and survive longer.
Introduction of fresh air and diagonal separation hot air curtain and UV curtain is activated
The customer enters and sits down, and the perimeter back curtains are activated too
Customer may order set the temperature of the central cold air, its humidity and flow speed to his preferences.
Customer is using communication system and screens to place order
Customer sets ambient light, and if the surrounding screens to be placed and the theme of images on the screens, sound surround and amore ambient details, as oxygen concentration, scents in air, etc.
For safety purposes, the booth system is taking customer's temperature
Food is delivered by a robotic shuttle, containing them in a pressurized and UV illuminated bay, and being delivered over the central barrier, at the prescribed temperature, and mass measured at delivery.
After eating, the payment is done by card or by phone, and when customer stands up the perimeter curtains are deactivated, while the nozzles are switched on negative pressure and absorb air.
A guiding light in the floor drives the customer outside the hall in a safe social-distancing area, and sequentially is treating all the other customers at the same table.
Interruptions, for restrooms or other emergencies when customers' needs to leave the place are treated in the same manner
After the table is free of customers, the decontamination procedure begins, by applying the shield, a robotic arm is cleaning the surfaces and temperature is increased at 140 f (50 C) and UV light is applied, and in some cases a Chlorine, Ozone, or other disinfectant gases may be applied, followed by rinse with clean air. Surfaces are painted with bio-active paints containing nano-particles of Ag, Ti, Ni, Pt, etc. that have an antiseptic effect.

For quality assurance purposes, at entry and exit customer's temperature is measured by IR camera, a mask is required to exit the hall, and foods, image, temperature and weight at delivery and return of dishes is recorded, customer receiving a bill with content of calories and ingredients to add at their dietary program. That measurement may be used or not to decline service for that customer group, but may be used only to assure customers that the location is safe.

As examples of applications depicted in figures one may see in FIG. 4 a module, as a constructive element of a table, and in FIG. 5 is shown how the 90 deg. prism booth may be used to produce a 4 seat table. If a group there may declare common air space and stop the barriers between them.

In FIG. 6 it is briefly presented how a 60-deg. prism booth may be used to produce a 6 seats hexagonal table, where a misplaced object crossing a barrier is detected and triggers an alarm, and stops that barrier in order to avoid any harm to customers.

FIG. 7 further shows a restaurant level formed by hexagonal tables having above ducts for robotic shuttle used for food delivery and dish withdrawal, as well for input and output air, water, sewage, etc.

FIG. 8 is a detail on technology and method used to assure protection that may be applied to any geometrical configuration, in a full new construction or as adaptation to the present furniture.

It was not clearly pictured, but it is obvious that this technology may be applied in mobile units as tents where in the cold season it may minimize need for heating, of the entire volume by maintaining at desired parameters the useful surface, say a bed, only.

FIG. 9 is an exemplification of this technology added to a cafeteria style table, where a half or the entire table may be protected.

FIG. 10 exemplifies how a table for two may be upgraded to protect the partners from mutual airborne droplets sharing, using the both airflow curtains to deflect and collect the exhaled plumes, and the UV as a supplementary measure, while using screens for full physical protection.

In FIG. 11 it is presented a pair of seats that may be on stadiums, theatres, conference rooms, airplanes, busses, trains that have the full protective system applied. In terms of seat compaction, it will not be possible to obtain pre-covid-19 performances where the customers to sit shoulder to shoulder of worse, because space for shield will be required, and that at minimum may take about ½ ft. (15 cm) with some physical barriers in the body zone to prevent disruption of the curtains.

FIG. 12 shows the enhancements one may have to bring to a bar table that to protect the customers against each-other and against bartender, keeping them safe from any bio-agent exchange.

This may be also applied to hospital beds, emergency tents or in any place where a bio-agent emitter is present and its insulation is desired, for example in a protective equipment (PPE) dressing and undressing room, where the agent is intentionally crossing the curtains, to gradually separate from the contaminated PPE, which in this process is partially sterilized and stored.

1. A method to stop and sterilize the bio-agents exhaled by a nearby person using:
a. An atmospheric box like structure formed of:
i. A fresh air intake with filtering and organic compound burning in catalytic filter;
ii. A carbon dioxide retention and ultrasound nebulizer;
iii. A downward going humid air current;
iv. A collector unit with a heater heating up the gas to 200 C;
v. A perimeter nozzle forming a hot ascending air curtain surrounding the central descending current;
vi. A top absorber nozzle that cleans and sterilizes the ascending current;
vii. A used gas discharge in atmosphere or partial recirculation;
b. A UV curtain on outer perimeter, outside the hot air curtain;
c. An advance detection of sneezing or coughing, which triggers a stopper using compressed air puffs and acoustic shock waves with constructive interference against the exhausted plume on its trajectory;
d. A system to sterilize the booth after customer's departure using UV light, sanitizer solution spray, and a hot air brush to dry up utilized on a robotic arm;
e. An optional digital display surrounding the customer(s) for added privacy, comfort, or preference;
f. A robotic shuttle food delivery system where the shuttle:
i. Delivers foods under positive pressure and UV light during transport on a delivery duct;
ii. Returns the dishes inside a negative pressure bay, with UV light using the return duct;

2. A method to stop and sterilize the bio-agents exhaled by a nearby person, according claim 1, where an air mist made by nebulizing water droplets with ultrasound, is used to create a virga effect to capture the exhaled gas where it is then absorbed under the customer and warmed up to 200 C (392 F) inside a heat exchanger. The heat is transported through a heat pipe from the evaporator of the heat-pump where an electric heater is used to assist the evaporator in gaining the necessary heat;

3. A method to stop and sterilize the bio-agents exhaled by a nearby person according claim 1, where the droplets exhaled and ambient air droplets are collected in a desiccator unit and vaporized inside a distiller boiling unit to kill the bio-agents, where the vapors are then sent up into a condenser to recycle the collected droplets;

4. A method to stop and sterilize the bio-agents exhaled by a nearby person according claim 1, where the UV curtain uses corner reflectors that return the beam several times in order to increase its efficiency;

5. A method to stop and sterilize the bio-agents exhaled by a nearby person according claim 1, where a catalytic burner is used to terminate all organic compounds before air enters in the intake duct;

6. A method to stop and sterilize the bio-agents exhaled by a nearby person according claim 1, where the products from the catalytic burner are filtered and carbon dioxide is removed by a sequestration device;

7. A method to stop and sterilize the bio-agents exhaled by a nearby person according claim 1, where the hot air curtains and UV curtain are protected against intrusions by an optical barrier and an image recognition and alert system;

8. A method to stop and sterilize the bio-agents exhaled by a nearby person according claim 1, that is versatile enough to be applied to various seating arrangements or table constructions;

9. A method to stop and sterilize the bio-agents exhaled by a nearby person according claim 1, where the food and/or drink order is delivered in a sterile manner under quality assurance conditions by a robotic shuttle, using dedicated paths of travel and access;

10. A seat assembly with enhanced individual protection comprising:
a. a modular structure forming a booth being made of:
i. compartments containing equipment above and below the customer's floor and ceiling;
ii. single wall structural support;
b. an ergonomic seat;
c. a sector of a table representing ¼ or ⅙ of a table, and a seat intended to be combined with other units to form a full table;
d. means to control airflow to form an atmospheric box inside the booth, made of:
i. air intake ducts;
ii. organic airborne fractions burner;
iii. carbon dioxide and other odors sequester unit;
iv. HEPA filter;
v. AC cooling unit;
vi. Humidifier using an ultrasound nebulizer;
vii. Airflow control fan and nozzle above the customer head;
viii. Air collector underneath customer;
ix. Air heater using a heat pipe and electric assisted heating;
x. Airflow control unit;
xi. Hot air boundary nozzles and section control;
xii. Hot air intake nozzles on ceiling;
xiii. Air cleaning and cooling for exhaust;
xiv. Used air pathway for exhaust or recycling;
e. Means to sterilize the air crossing the airflow curtains using UV light
f. Means of protecting the customer when crossing the protective curtains made of:
i. Optical barrier;
ii. Image detection of trespassing;
iii. Alarm system;
iv. Specific barrier curtain shutdown system;
g. Means to protect the opposed customers against unexpected coughing or sneezing comprising:
i. Sound detection and identification system of cough or sneeze;
ii. Compressed air system with sudden release upon detection, forming a directed jet-puff;
h. Means to save energy, by using heat pipes and/or heat pumps, air-air heat exchangers, and assisted electric heating;
i. Increased privacy or comfort system composed off digital screens with image and noise suppression and sound surround which can seal the table or table formation;
j. Modular structure that allows fast reconfiguration in a large variety of different formations;
k. Capabilities of delivering a gas therapy or thermal therapy to customers;
l. Capabilities of having a robotic order delivery using a shuttle that utilizes both pressure and UV irradiation to increase safety and sterilization;
m. Means to automatic cleaning and sterilization that comprises:
i. An air tight sealing system using plastic or digital screens;
ii. Capability of applying septic gases, without leakage;
iii. A robotic arm disinfecting surfaces with a chemical shower and brush;
iv. Active paint with self-sterilization capability activated by UV light;
v. Gas rinsing with oxygen and nebulized air;
n. Capability or communication payment and ordering;
o. Capability to vary the booth content and shape to be the elemental cell of various configurations as:
i. Restaurant tables for a variable number groups;
ii. Bar tables;
iii. Seats only, for various applications as meeting rooms, seminar rooms, airplanes, trains, stadiums;
p. Capability of providing quality assurance using means as:
i. Customer temperature measurements at entry and departure;
ii. Food parameters measurement;
iii. List of contents of the treatment applied describing: food; drinks; deserts; other shopping; gas; privacy theme;

11. A seat assembly with enhanced individual protection according to claim 10, where a seat only is surrounded by the protective hot air curtain, UV curtain, and customer protection optical barrier;

12. A seat assembly with enhanced individual protection according to claim 10, where hot air and UV barriers are applied all around on the boundaries and they are activated and deactivated upon request;

13. A seat assembly with enhanced individual protection according to claim 10, where the conditioned air is made by using both an AC and ultrasound nebulizer to produce a mist that evaporates before reaching the customer, creating a downward airflow that captures the exhaled gasses;

14. A seat assembly with enhanced individual protection according to claim 10, where customers are protected from each other and surroundings for any germs or viruses exchange by a redundant system of toroidal convection currents and UV curtains acting as individual shields;

15. A seat assembly, with enhanced individual protection according to claim 10, where sudden unexpected incidents or air-droplets exchange incidents are denied by using an advanced detection, air puff generators create a current acting with constructive interference;

16. A seat assembly with enhanced individual protection according to claim 10, using plastic or digital screens to physically shield the booth, for increased privacy, comfort, preference, or for cleanup after customers;

17. A seat assembly with enhanced individual protection according to claim 10, applied to individual seats for airplane, trains, stadiums, conference rooms where the table for serving food is missing;

18. A seat assembly with enhanced individual protection according to claim 10, that uses various gases and scents to deliver a private and personalized theme environment;

19. A seat assembly with enhanced individual protection according to claim 10, which is used to deliver safe food provided by robotic shuttles with increased sterilization capabilities through the use of gas under controlled pressure and UV irradiation, following quality assurance protocols;

20. A seat assembly with enhanced individual protection according to claim 10, that uses a specialized sterile surface coating, with UV active structures, and a robotic arm to clean surfaces of interest between customers.
